(19) United States
(12) Patent Application Publication
(10) Pub. No.: US 2024/0299472 A1
(43) Pub. Date: Sep. 12, 2024

(54) COMPOSITION FOR INCREASING MUSCLE MASS AND SUPPRESSING A DECREASE IN MUSCLE MASS, AND COMPOSITION FOR INCREASING MUSCLE MASS AND SUPPRESSING MUSCLE ATROPHY

(71) Applicant: DIC Corporation, Tokyo (JP)

(72) Inventors: Yasuyuki IMAI, Chiba (JP); Yurino KOSEKI, Chiba (JP)

(73) Assignee: DIC Corporation, Tokyo (JP)

(21) Appl. No.: 18/273,779

(22) PCT Filed: Jan. 20, 2022
(86) PCT No.: PCT/JP2022/001881

§ 371 Date: Jul. 24, 2023

(30) Foreign Application Priority Data
Jan. 25, 2021 (JP) 2021-009310

(51) Int. Cl. A61K 35/748; A61K 31/409; A61P 21/00

(52) U.S. Cl. CPC A61K 35/748; A61K 31/409; A61P 21/00

(57) ABSTRACT

The present invention addresses the problem of providing a composition for increasing muscle mass and suppressing a decrease in muscle mass, and composition for increasing muscle mass and suppressing muscle atrophy, the compositions can be taken orally continuously in a daily basis. As a result of intensive research, the inventors solved the problem by preparing a composition for increasing muscle mass and suppressing a decrease in muscle mass, which contains as an effective component at least one selected from the group consisting of spirulina, phycocyanin, a spirulina enzymatically degraded product, and a phycocyanin enzymatically degraded product.

### TECHNICAL FIELD

The present invention relates to a composition for increasing muscle mass and suppressing a decrease in muscle mass and a composition for increasing muscle mass and suppressing muscle atrophy.

### BACKGROUND ART

In recent years, decreases in muscle strength and decreases in muscle mass associated with aging are called “sarcopenia”, and a treatment method therefor has attracted attention with increasing patients. The sarcopenia is accompanied by a decrease in skeletal muscle mass, but in a broad sense, it is considered to include ones accompanied by a decrease in skeletal muscle strength and a decrease in physical function. The sarcopenia is considered to be associated with various factors such as nutrient intakes, hormones, inflammatory reaction, etc. other than decreases in physical activity which changes with aging. When patients are once affected with sarcopenia, a vicious circle of easy fall/fall down, fracture, body movement limitation, and progression of sarcopenia is exhibited, causing a bedridden state.

At present, it is considered that muscle strength training is effective for preventing and curing sarcopenia, but a high-strength exercise intervention is difficult for elderly persons having complications in joints, circulatory organs, or the like and for elderly persons already forced to have physical movement limitation. Therefore, a food and/or medicine, which can be ingested continuously orally on a daily basis without a high-strength exercise intervention, is desired to be developed for preventing and curing sarcopenia. An essential amino acid, hormone drug, ACE inhibitor, and the like are investigated as a food and/medicine for preventing and curing sarcopenia, but there are still not many foods and/or medicines exhibiting the sufficient effect on sarcopenia.

Also, due to recent increases in health consciousness, spirulina attracts attention as a food having an excellent nutrient balance and a higher nutritional value than general foods. Spirulina is a cyanobacteria species containing rich protein, saccharides, various vitamins, minerals, and plant pigments. Spirulina is used as a food or supplement for taking various types of nutrients in a good balance, and spirulina itself and spirulina-derived materials are expected to have many functionalities. Therefore, researches about the use of spirulina are actively performed.

For example, a lipase activity inhibitor containing phycocyanin as an effective component is reported because spirulina-derived phycocyanin has the function of inhibiting the activity of lipase such as pancreatic lipase (refer to Patent Literature 1).

Also, a serum-lipid reducing agent containing phycocyanin as an effective component is reported because spirulina-derived phycocyanin has a higher serum-lipid improving function than soybean protein (refer to Patent Literature 2).

Further, it is reported that the activation of natural killer (NK) cells is induced by oral administration of spirulina (spirulina extract) (refer to Non Patent Literature 1).

### CITATION LIST

### Patent Literature

PTL 1: Japanese Unexamined Patent Application Publication No. 2004-359638
PTL 2: Japanese Unexamined Patent Application Publication No. 2003-137805

### Non Patent Literature

NPL 1: Hirahashi T, et al. “Activation of the human innate immune system by spirulina: Augmentation of interferon gamma production and NK cytotoxicity by oral administration of spirulina.” International Immunopharmacology 2 (2002) 423-434.

### SUMMARY OF INVENTION

### Technical Problem

However, it has not been known so far that spirulina itself and a spirulina-derived material exhibit the function of increasing muscle mass, the function of suppressing a decrease in muscle mass, and the function of suppressing muscle atrophy.

An object of the present invention is to provide a composition for increasing muscle mass and suppressing a decrease in muscle mass and a composition for increasing muscle mass and suppressing muscle atrophy, the compositions being possible to be ingested continuously orally on a daily basis.

### Solution to Problem

As a result of earnest research for solving the problem, the inventors found that spirulina itself and a spirulina-derived material have the function of increasing muscle mass, the function of suppressing a decrease in muscle mass, and the function of suppressing muscle atrophy, leading to the achievement of the present invention.

That is, the present invention includes the following aspects.

[1] A composition for increasing muscle mass and suppressing a decrease in muscle mass, which contains as an effective component at least one selected from the group consisting of spirulina, phycocyanin, a spirulina enzymatically degraded product, and a phycocyanin enzymatically degraded product.

[2] The composition described in [1], in which the enzyme for the spirulina enzymatically degraded product and the phycocyanin enzymatically degraded product is protease.

[3] A composition for increasing muscle mass and suppressing muscle atrophy, which contains as an effective component at least one selected from the group consisting of spirulina, phycocyanin, a spirulina enzymatically degraded product, and a phycocyanin enzymatically degraded product.

[4] The composition described in [3], in which the enzyme for the spirulina enzymatically degraded product and the phycocyanin enzymatically degraded product is protease.

[5] The composition described in any one of [1] to [4], which is a food composition.

[6] The composition described in [5], which is a health food, a functional food, a nutritional supplement food, a supplement, a food with a health claim, a food for specialized health uses, a functional nutritional food, a food with function claim, or a food for patients.

[7] The composition described in any one of [1] to [4], which is a pharmaceutical composition.

[8] The composition described in [7], which is a pharmaceutical composition used for curing, preventing, or improving sarcopenia or locomotive syndrome.

The present invention can provide a composition for increasing muscle mass and suppressing a decrease in muscle mass and a composition for increasing muscle mass and suppressing muscle atrophy, the compositions being possible to be ingested continuously orally on a daily basis.

### BRIEF DESCRIPTION OF DRAWINGS

FIG. 1 is a graph showing the proportions of expression levels of genes estimated to be associated with the increase muscle mass and the suppression of a decrease in muscle mass of the gastrocnemius muscle relative to an A1 group (Sham group) in test 1.

FIG. 2 is a graph showing the proportions of expression levels of genes estimated to be associated with muscle atrophy in the gastrocnemius muscle relative to an A1 group (Sham group) in test 1.

### DESCRIPTION OF EMBODIMENTS

A composition according to an embodiment of the present invention is described in detail below, but the description of constituent features below is an example of an embodiment of the present invention, and the present invention is not limited to the contents thereof.

A composition according to an embodiment of the present invention relates to a composition for increasing muscle mass and suppressing a decrease in muscle mass, which contains as an effective component at least one selected from the group consisting of spirulina, phycocyanin, a spirulina enzymatically degraded product, and a phycocyanin enzymatically degraded product.

Also, a composition according to an embodiment of the present invention relates to a composition for increasing muscle mass and suppressing muscle atrophy, which contains as an effective component at least one selected from the group consisting of spirulina, phycocyanin, a spirulina enzymatically degraded product, and a phycocyanin enzymatically degraded product.

The effective components of a composition according to an embodiment of the present invention are described below, and then the uses, forms etc. of the composition are described.

### <Spirulina>

Spirulina is an effective component of the composition according to the embodiment and has the function of increasing muscle mass, the function of suppressing a decrease in muscle mass, and the function of suppressing muscle atrophy.

Spirulina is a minute spiral alga belonging to the class Cyanobacteria, the order Nostocales, the family Oscillatoriaceae, the genus Spirulina, and contains rich protein, saccharides, various vitamins, minerals, and plant pigments.

Examples of spirulina include Spirulina platensis, Spirulina maxima, Spirulina geitleri, Spirulina siamese, Spirulina major, Spirulina subsalasa, Spirulina princeps, Spirulina laxissima, Spirulina curta, Spirulina spirulinoides, and the like. Among these, examples of spirulina particularly preferred because it can be artificially cultured and is easily available include Spirulina platensis, Spirulina geitleri, Spirulina Siamese, and the like. In addition, spirulina is also referred to as “Arthrospira”.

An alga (wet alga) in a state of being cultured in a liquid culture medium may be directly used as the spirulina used in the composition according to the embodiment, but it is preferred to use a spirulina extract which is an extracted solution obtained by extracting the wet alga of spirulina with a solvent such as water, ethanol, or the like, or an extract obtained by concentrating or drying the extracted solution.

### <<Spirululina Extract>>

The extracted solution used for producing the spirulina extract is not particularly limited within a range in which the effect of the present invention can be obtained, but for example, hot water can be used. In the embodiment, an extracted solution obtained by extracting the wet alga of spirulina with hot water or an extract obtained by concentrating or drying the extracted solution can be preferably used.

A method for producing the spirulina extract is not particularly limited, and the extract can be produced according to a general method. Examples thereof include the method for producing an extracted solution described in Non Patent Literature 1 and Japanese Unexamined Patent Application Publication No. 8-9940, and the like. Specifically, a production method described below can be given.

### <<Method for Producing Spirulina Extract>>

The spirulina extract can be produced by extracting the spirulina alga with hot water at a temperature exceeding 100° C., adjusting the pH of the resultant extracted solution under specified acid conditions, and then removing an insoluble fraction, producing a spirulina extracted solution.

When the spirulina extract is used in a liquid state, the spirulina extracted solution obtained described above can be directly used. Also, the spirulina extracted solution can be used as a powder after concentration or drying. The spirulina extract used in the composition according to the embodiment may be the spirulina extracted solution or may be produced by concentrating or drying the spirulina extracted solution.

The spirulina used for producing the spirulina extract may be a commercial product or may be cultured by oneself. Also, spirulina in a raw state may be used. The spirulina in a raw state may be dried and then used.

The spirulina can be cultured by a culture method according to a general method used for culturing cyanobacteria. For example, spirulina can be cultured and proliferated under basic conditions outdoors.

The spirulina (also referred to as the “spirulina alga”) obtained by culture can be used directly or the cultured spirulina can be recovered by filter cloth or filter paper, washed with water, and then suspended in water, preparing a suspension. Further, the culture solution or the suspension may be concentrated to prepare a wet alga. The wet alga may be dried by freeze drying, spray drying (spray-drying), or the like to prepare a dried alga, and the dried alga may be powdered.

The spirulina alga used in hot water extraction may be any one of a wet alga, a freeze-dried alga, a spray-dried alga, a crushed alga, and the like. For example, the crushed alga can be obtained by, for example, crushing treatment of the alga using a general method, for example, an industrial high-pressure extrusion method using a French press or the like.

Next, a hot water extraction operation is described. For example, the spirulina alga processed as described above is previously suspended in an extraction solvent, for example, distilled water, in a pressure vessel. The suspension concentration is not particularly limited, but considering the extraction efficiency, recovery cost, etc., it is preferably 1% to 20% by mass relative to the solvent. The extraction solvent may be tap water but considering the point that the extracted solution is used as a food raw material, distilled water is preferred. The extraction temperature is generally a temperature exceeding 100° C., preferably 105° C. to 140° C., and more preferably 110° C. to 130° C. The pressure for extraction is preferably 1.0 to 2.5 atm. During extraction, a stirring operation may be or may not be performed, but the stirring operation is preferably performed in view of thermal efficiency. The extraction amount is increased with increasing extraction time, but the extraction time is generally preferably 0.5 to 4 hours in view of efficiency.

Next, the alga residue and the aggregated protein formed by thermal denaturation are removed from the alga residue suspension (pH=about 6.8 to 7.0) after the extraction operation. The removal operation may be, for example, an operation of centrifuging or filtrating the suspension or the like, and a supernatant is obtained by the operation. However, the supernatant still contains a large amount of protein dissolved therein, and thus the dissolved protein is preferably further removed. In order to further removed the dissolved protein, an acid is preferably added to the suspension to adjust the pH of the extracted solution to an acid condition equal to or lower than the isoelectric point of the protein. Consequently, the protein is aggregated, and the spirulina extract can be obtained by separating the aggregated protein by centrifugation, filtration, or the like. Alternatively, the pH of the alga residue suspension after the extraction operation may be adjusted to be equal to or lower than the isoelectric point of the protein as described above without first removing the alga residue etc., and then the alga residue and the aggregated protein may be separated by centrifugation, filtration, or the like in the same manner as described above. The removal of the aggregated protein can produce the spirulina extract containing a high yield of saccharides.

The acid condition equal to or lower than the isoelectric point of protein is preferably pH 4.5 or less, more preferably pH 3.75 to 4.25 because the aggregation and precipitation of protein are maximized, and is still more preferably pH 4.0.

The acid added for adjusting pH may be sulfuric acid or hydrochloric acid, but considering the practical working process and the use as a food material, an organic acid such as citric acid, malic acid, or the like is more preferably used than an inorganic acid.

### <Phycocyanin>

Phycocyanin is an effective component in the composition according to the embodiment and has the function of increasing muscle mass, the function of suppressing a decrease in muscle mass, and the function of suppressing muscle atrophy.

Phycocyanin is a chromoprotein and has phycocyanobilin as a chromophore. Phycocyanin has a structure in which phycocyanobilin and protein are bonded together.

Examples of phycocyanin used in the composition according to the embodiment include algae-derived phycocyanin such as cyanobacteria-derived phycocyanin, red algae-derived phycocyanin, cryptophyte-derived phycocyanin, and the like. Among these, cyanobacteria-derived phycocyanin is preferred because it can be collected in a large amount.

Examples of cyanobacteria include cyanobacteria of the genus Spirulina, the genus Arthrospira, the genus Aphanizomenon, the genus Fisherella, the genus Anabaena, the genus Nostoc, the genus Synechocystis, the genus Synechococcus, the genus Tolypothrix, the genus Aphanothece, the genus Mastigoclaus, the genus Pleurocapsa, and the like. Among these, cyanobacteria of the genus Spirulina and the genus Arthrospira are preferred because they are produced on an industrial scale and the safety thereof is confirmed, and cyanobacteria of the genus Spirulina are more preferred.

Also, raw cyanobacteria may be used as a raw material for preparing phycocyanin, or dried cyanobacteria after drying may be used. A dried product of cyanobacteria may be a dried product produced from raw cyanobacteria according to a general method, or a commercial dried product may be used.

Examples of phycocyanin include C-phycocyanin, R-phycocyanin, allophycocyanin, and the like. From the viewpoint of quality, safety, or easy availability, or the like, C-phycocyanin is preferably contained as phycocyanin. Therefore, in a preferred mode, the composition according to the embodiment is a composition containing C-phycocyanin as an effective component. Further, in a preferred mode, the composition according to the embodiment is a composition containing C-phycocyanin and allophycocyanin. For example, a phycocyanin mixture of C-phycocyanin and allophycocyanin, obtained by extraction from cyanobacteria of the genus Spirulina, can be contained in the composition.

The phycocyanin can be obtained by, for example, suspending cyanobacteria in water or a buffer solution such as a phosphate buffer solution, a citrate buffer solution, or the like, and then extracting phycocyanin in the cyanobacteria.

A method for extracting phycocyanin is not particularly limited, and phycocyanin can be extracted according to a general method.

Examples of a preferred mode of the extraction method include the extraction method described in Japanese Unexamined Patent Application Publication No. 2006-230272, and the like. Specifically, an extraction method described in extraction method (i) below is given. The extraction method (i) can produce phycocyanin having high purity and a bright color.

### <Extraction Method (i) for Phycocyanin>

The extraction method (i) includes:
a first step of obtaining an extracted solution by extracting phycocyanin in cyanobacteria into a water suspension;
a second step of producing calcium phosphate by reacting a calcium salt and a phosphate salt in the extracted solution and adsorbing impurities of phycocyanin on the calcium phosphate to obtain an adsorbed substance; and
a third step of removing the residue of cyanobacteria and the adsorbed substance from the extracted solution.

The extraction method (i) described above is preferably an extraction method (ii) below.

### <<Extraction Method (ii) for Phycocyanin>>

The extraction method (ii) includes:
a first step of obtaining an extracted solution by extracting phycocyanin in cyanobacteria into a water suspension;
a second step of producing calcium phosphate by reacting a calcium salt and a phosphate salt in the extracted solution and adsorbing impurities of phycocyanin on the calcium phosphate to obtain an adsorbed substance;
a third step of removing the residue of cyanobacteria and the adsorbed substance from the extracted solution; and
a step of allowing the extracted solution to contain a chelating agent before the third step.

The phycocyanin having high quality can be extracted from cyanobacteria by using the extraction method (i) or (ii) for phycocyanin.

In particular, when the extraction method (i) or (ii) is used for cyanobacteria of the genus Spirulina, phycocyanin having a good mixing ratio between C-phycocyanin and allophycocyanin and good quality can be extracted.

In addition, the mixing ratio between C-phycocyanin and allophycocyanin is preferably adjusted to be within a desired range by appropriately selecting the extraction conditions.

The types of phycocyanin are all C-phycocyanin.

Alternatively, allophycocyanin may be contained, and a mixture of C-phycocyanin and allophycocyanin may be contained in the composition.

The mixing ratio between C-phycocyanin and allophycocyanin is, for example, preferably a mass ratio of 3 to 9.5:0.5 to 7, more preferably 6 to 9.5:0.5 to 4, and still more preferably 7 to 8:2 to 3.

### <Spirulina Enzymatically Degraded Product>

The spirulina enzymatically degraded product is an effective component of the composition according to the embodiment, and has the function of increasing muscle mass, the function of suppressing a decrease in muscle mass, and the function of suppressing muscle atrophy.

The spirulina enzymatically degraded product is an enzymatically degraded product obtained by enzyme action to decompose the protein, saccharide, etc. of spirulina described above.

Usable examples of the enzyme used for enzymatic degradation include, but are not particularly limited to, glucanase, kinase, protease, pectinase, lipase, cellulase, xylanase, mannanase, hemicellulase, nuclease, and the like. Among these, from the viewpoint that decomposition of spirulina is preferably performed, protease is preferred as the enzyme used for enzymatic degradation. The enzymes can be used alone or in combination of two or more.

Usable examples of the origin of the enzyme used for enzymatic degradation include, but are not particularly limited to, Aspergillus niger, Aspergillus melleus, Aspergillus oryzae, Rhizopus niveus, Bacillus subtilis, Arthrobacter sp., Trichoderma viride, and the like.

The protease is not particularly limited, and a commercial protease formulation can be used. Usable examples of the protease formulation include Sumizyme LP, Sumizyme FL-G, Sumizyme CP, Sumizyme FP-G, and Sumizyme MP (Shin Nihhon Chemical Co., Ltd.), Bromerain F, Protease P “Amano” 3SD, Papain W-40, Thermoase PC10F, Thermoase C100, Thermoase C160, and Protin SD-NY10 (Protin SD-PC10F) (Amano Enzyme Inc.), and the like.

In the molecular weight distribution of proteins in the spirulina enzymatically degraded product, the ratio of components having a molecular weight of less than 500 is preferably 20% by mass or more, more preferably 30% by mass or more, and still more preferably 35% by mass or more. When the molecular weight distribution of proteins in the spirulina enzymatically degraded product is equal to or higher than the lower limit described above, the spirulina enzymatically degraded product has excellent in vivo absorbability.

The value of the molecular weight distribution can be obtained by liquid chromatography analysis of a sample using a gel filtration column.

The treatment method for enzymatic degradation is not particularly limited, and treatment can be performed according to a general method. Also, the reaction conditions of enzymatic degradation are not particularly limited, and the reaction conditions such as the optimum temperature, optimum pH, reaction time, etc. may be properly adjusted according to the type, activity, amount of the enzyme added and the like. The spirulina enzymatically degraded product obtained by enzymatic degradation treatment can be adjusted to the intended form and properties by operations such as centrifugation, filtration, desalting, concentration, drying, solvent extraction, dilution, and the addition of additives, and the like.

An example of the method for preparing the spirulina enzymatically degraded product in the composition according to the embodiment is described below.

An aqueous spirulina solution is heated at 20° C. to 70° C. The aqueous solution is adjusted to the optimum pH for the enzyme used, and then 0.01% by mass or more, preferably 0.1% to 10% by mass, of the enzyme is added relative to the protein in spirulina, followed by stirring for 1 to 24 hours. After stirring, deactivation/stop of enzymatic degradation is performed by heating or cooling, followed by centrifugation. After centrifugation, the resultant supernatant is freeze-dried, thereby obtaining the spirulina enzymatically degraded product.

### <Phycocyanin Enzymatically Degraded Product>

The phycocyanin enzymatically degraded product is an effective component in the composition according to the embodiment and has the function of increasing muscle mass, the function of suppressing a decrease in muscle mass, and the function of suppressing muscle atrophy.

The phycocyanin enzymatically degraded product is an enzymatically degraded product obtained by enzyme action to decompose the phycocyanin described above.

The enzyme used for enzymatic degradation is not particularly limited, and the enzymes described above for the spirulina enzymatically degraded product can be used. Therefore, the description thereof is omitted. In addition, like in the spirulina enzymatically degraded product, protease is preferably used as the enzyme used for enzymatic degradation.

In the molecular weight distribution of the phycocyanin enzymatically degraded product, the ratio of components having a molecular weight of less than 500 is preferably 20% by mass or more, more preferably 30% by mass or more, and still more preferably 35% by mass or more. When the molecular weight distribution of the phycocyanin enzymatically degraded product is equal to or higher than the lower limit described above, the phycocyanin enzymatically degraded product has excellent in vivo absorbability.

The value of the molecular weight distribution can be obtained by liquid chromatography analysis of a sample using a gel filtration column.

The protease used for enzymatic degradation and the treatment method for enzymatic degradation are not particularly limited and are the same as described above for the spirulina enzymatically degraded product, and thus the description thereof is omitted.

An example of the method for preparing the phycocyanin enzymatically degraded product in the composition according to the embodiment is described below.

An aqueous phycocyanin solution is heated at 20° C. to 70° C. The aqueous solution is adjusted to the optimum pH for the enzyme used, and then 0.01% by mass or more, preferably 0.1% to 10% by mass, of the enzyme is added relative to phycocyanin, followed by stirring for 1 to 24 hours. After stirring, deactivation/stop of enzymatic degradation is performed by heating or cooling, followed by centrifugation. After centrifugation, the resultant supernatant is freeze-dried, thereby obtaining the phycocyanin enzymatically degraded product.

### <Composition for Increasing Muscle Mass and Suppressing a Decrease in Muscle Mass>

As shown in examples described below, the effective components such as spirulina, phycocyanin, the spirulina enzymatically degraded product, and the phycocyanin enzymatically degraded product were recognized to alleviate the suppression of expression of the gene estimated to be associated with the increase in muscle mass and the suppression of a decrease in muscle mass. Examples of the gene of which the suppression of expression is alleviated by the effective components and which is estimated to be associated with the increase in muscle mass and the suppression of decrease in muscle mass include Myh13 (Myosin, heavy chain 13), Myh2 (Myosin, heavy chain 2), My12 (Myosin, light polypeptide 2), Myh6 (Myosin, heavy chain 6), Tnni1 (Troponin I type 1), Tnnc1 ((Troponin C type 1), Itm2a (Integral membrane protein 2A), and Neu2 (Neuraminidase 2), and the like.

Therefore, the composition according to the embodiment can be used as a composition for increasing muscle mass and suppressing a decrease in muscle mass, which contains as an effective component at least one selected from the group consisting of spirulina, phycocyanin, the spirulina enzymatically degraded product, and the phycocyanin enzymatically degraded product.

The muscle as a target of the increase in muscle mass and the suppression of decrease in muscle mass is not particularly limited, but is preferably skeletal muscle. The skeletal muscle is considered to be associated with knee-joint extension and hip-joint bending, specifically, the motion of extending the knee and standing up, walking motion, running motion, and the like. An increase in skeletal muscle mass improves the motions of standing-up, walking, running, and the like, and is considered to be particularly effective for curing, preventing, or improving sarcopenia and locomotive syndrome.

The locomotive syndrome represents a syndrome in a state where a trouble is caused in a locomotorium part such as muscle, bone, or the like due to aging, a lack of exercise, or the like, leading to difficulty in daily lives. The locomotive syndrome is a concept including the syndromes of the locomotorium in general, while sarcopenia is regarded as the syndrome particularly focused on muscle mass, muscle strength, and physical functions in the locomotorium.

Examples of the skeletal muscle include sternocleidomastoideole, greater pectoral muscle, smaller pectoral muscle, anterior serratus muscle, subclavius muscle, abdominal rectus muscle, abdominal external oblique muscle, abdominal internal oblique muscle, transverse abdominal muscle, quadratus lumborum muscle, trapezius muscle, Latissimus dorsi muscle, erector spinae muscle, levator scapulae muscle, rhomboideus muscle, deltoid muscle, teres minor muscle, supraspinatus muscle, infraspinatus muscle, subscapularis muscle, teres major muscle, coracobrachial muscle, biceps brachii muscle, brachial muscle, brachioradialis muscle, triceps brachii muscle, anconeus muscle, pronator teres muscle, quadrate pronator muscle, supinator muscle, flexor carpi ulnaris muscle, flexor carpi radialis muscle, palmaris longus muscle, flexor digitorum superficialis muscle, flexor digitorum profundus muscle, Flexor hallucis longus muscle, extensor carpi radialis longus muscle, extensor carpi radialis brevis muscle, extensor carpi ulnaris muscle, extensor digitorum muscle, extensor indicis muscle; extensor digiti minimi muscle, extensor pollicis longus muscle, extensor pollicis brevis muscle, abductor pollicis longus muscle, lumbricals muscle (four muscles), interossei palmares muscle (three muscles), interossei dorsales muscle (four muscles), abductor digiti minimi muscle, flexor digiti minimi brevis muscle, opponens digiti minimi muscle, palmaris brevis muscle, adductor pollicis muscle, flexor pollicis brevis muscle, opponens pollicis muscle, abductor pollicis brevis muscle, rectus femoris muscle, lateral muscle, intermediate muscle, vastus medialis muscle, iliacus muscle, psoas major muscle, psoas minor muscle, sartorius muscle, pectineus muscle, tensor fasciae latae muscle, gluteus maximus muscle, biceps femoris, quadriceps femoris, semitendinosus muscle, semimembraneous muscle, gluteus medius muscle, gluteus minimus muscle, gracilis muscle, adductor longus muscle, adductor brevis muscle, adductor magnus muscle, six deep external rotator muscles, gastrocnemius muscle, soleus muscle, popliteus muscle, tibialis posterior muscle, flexor digitorum longus muscle, flexor hallucis longus muscle, plantaris muscle, anterior tibial muscle, fibularis longus muscle, fibularis brevis muscle, peroneus tertius muscle, extensor hallucis longus muscle, extensor digitorum longus muscle, and the like.

### <Composition for Increasing Muscle Mass and Suppressing Muscle Atrophy>

As shown in examples described below, the effective components such as spirulina, phycocyanin, the spirulina enzymatically degraded product, and the phycocyanin enzymatically degraded product were recognized to alleviate the enhancement of expression of the gene estimated to be associated with muscle atrophy. Examples of the gene of which the enhancement of expression is alleviated by the effective components and which is estimated to be associated with muscle atrophy include Ddit4 (DNA-damage-inducible transcript 4), Junb (Jun B proto-oncogene), Egr1 (zif-268, Early growth response protein 1, Sdc4 (Syndecan 4), Kcnk5 (Potassium channel, subfamily K, member 5), Rasd2 (Rhes, RASD family, member 2), and the like.

Therefore, the composition according to the embodiment can be used as a composition for increasing muscle mass and suppressing muscle atrophy, which contains as an effective component at least one selected from the group consisting of spirulina, phycocyanin, the spirulina enzymatically degraded product, and the phycocyanin enzymatically degraded product.

The muscle atrophy represents a decrease in muscle mass due to a decrease or reduction of muscle cells, and examples thereof include that caused by long-term bed rest, fixation by plaster cast due to fracture, or the like, that caused by microgavity exposure (disuse muscle atrophy), that with increasing age (also referred to as “aging”), and the like. Therefore, the suppression of muscle atrophy represents that the suppression of a decrease in muscle mass with inactivity or aging.

The muscle as an object of suppression of increase in muscle mass and of muscle atrophy is the same as that described above for the composition for increasing muscle mass and suppressing a decrease in muscle mass, and thus the description thereof is omitted.

### (Food Composition)

The composition according to the embodiment can be provided, in the food field, as a general food and also as a food composition having an intended function when an effective amount of an effective component, which can effectively exhibit the intended function, is mixed as a food material in various foods. The composition according to the embodiment can be preferably used for food compositions, for example, a health food, a functional food, a nutritional supplement food, a supplement, a food with a health claim, a food for specialized health uses, a functional nutritional food, a food with function claim, a food for patients, a food additive, a feed, a feed additive, and the like. The form of the food composition is not particularly limited and can be properly selected according to purpose, and examples thereof include a solid form, a liquid form, a gel form, and the like.

In addition, the food with function claim is a food which displays functionality based on scientific evidence under the responsibility of business operators, and a food with the information, about the evidence of safety and functionality, submitted to the Secretary-General of the Consumer Affairs Agency before sales. The food composition according to the embodiment may have a display, as a food composition for the purpose of increasing muscle mass and suppressing a decrease in muscle mass and/or increasing muscle mass and suppressing muscle atrophy, that “it is useful for suppressing a decrease in muscle mass and muscle strength required for living an independent daily life”, “it is useful for improving the walking ability”, “it maintains muscle mass and muscle strength”, “it supports the maintenance of muscle mass and muscle strength”, “it maintains the walking ability weakened by aging in middle-aged and older persons”, “it supports the force to build the muscle useful for maintaining the muscle weakened by aging”, or the like.

Examples of the food composition include non-alcoholic drinks such as soft drinking water, a carbonated drink, a fruit juice drink, a vegetable juice drink, a fruit juice and vegetable juice drink, animal milk such as bovine milk, soy milk, a milk-based drink, drink-type yogurt, a drink-type or stick-type jelly, coffee, cocoa, a tea drink, a nutrient drink, an energy drink, a sport drink, mineral water, near-water, a non-alcoholic beer-taste drink, and the like; carbohydrate-containing drinks/foods such as rice, noodle, breads, pastas, and the like; milk products such as cheeses, hard-type or soft-type yogurt, fresh cream from animal milk or other oil and fat raw materials, ice cream, and the like; cake and confectioneries such as western confectioneries, such as cookie, cake, chocolate, and the like, Japanese sweets such as a sweet bean cake, a sweet bean jelly, and the like, tablet sweets (refreshing sweets), Ramune soda and the like, candies, gums, ice confection and frozen sweets such as jelly, pudding, and the like, snacks, and the like; alcoholic drinks such as whiskey, bourbon, spirits, liqueur, wine, fruit wine, rice wine, Chinese alcohol, Japanese shochu, beer, non-alcohol beer with an alcohol percentage of 1% or less, law malt beer, other miscellaneous liquors, shochu highball, and the like; a processed product using egg, processed products (including delicacies) of seafood and meat (including offal such as lever and the like), processed foods such as soups, such as miso soup and the like, miso, soy sauce, Furikake, other seasonings, and the like, liquid diets such as concentrated liquid diet and the like; and the like.

Also, when the food composition is a health food, a functional food, a nutritional supplement food, a supplement, a food with a health claim, a food for specialized health uses, a functional nutritional food, a food with function claim, a food for patients, a food additive, a feed, a feed additive, and the like, the composition may be a tablet (including a chewable tablet or the like), a capsule, a troche, a syrup, jelly, a granule, a powder, or the like.

In addition to the effective components described above, one or more components freely selected from components, which can be used for general food compositions, may be mixed in the food composition according to the embodiment. For example, all additives which can be generally used in the food field, such as various seasonings, an antiseptic agent, an emulsifier, a stabilizer, a spice, a coloring agent, a preservative, a pH adjuster, and the like, can be contained.

### (Pharmaceutical Composition)

The composition according to the embodiment can be provided, in the medicine field, as a pharmaceutical composition having an intended function when mixed with a pharmaceutically acceptable carrier, additive, and the like together with an effective amount of an effective component, which can effectively exhibit the intended function. For example, the composition according to the embodiment can be preferably used as a pharmaceutical composition for curing, preventing, or improving the sarcopenia or locomotive syndrome described above. The pharmaceutical composition may be either a medical product or a nonmedicinal product.

The form of the pharmaceutical composition is not particularly limited and can be properly selected according to purpose, and examples thereof include a solid form, a liquid form, a gel form, and the like.

The pharmaceutical composition can contain additives such as a pharmaceutically acceptable general carrier, a binder, a stabilizer, an excipient, a diluent, a pH buffer, a disintegrating agent, a solubilizer, a solution aid, a tonicity agent, and the like. The pharmaceutical composition may be used for either oral or parenteral administration, but is more preferably used for oral administration. In use for oral administration, examples of a commonly used administration forms include administration in the dosage form of a tablet, a powder, a granule, a capsule, a sirup, a suspension, or the like. In use for parenteral administration, examples of a commonly used administration form include injection (subcutaneous injection, intravenous injection, intramuscular injection, or the like) in the dosage form of a solution, an emulsion, a suspension, or the like, intranasal administration in the dosage form of a spray, and the like.

Described below is an examples of a case in which the form of the food composition and/or the pharmaceutical composition is a tablet.

When the form of the food composition and/or the pharmaceutical composition is, for example, a tablet, the composition can be prepared by properly combining the effective components with additives such as an excipient, a binder, a disintegrating agent, a lubricant, a preservative, an antioxidant, a tonicity agent, a buffer agent, a coating agent, a flavoring agent, a solubilizer, a base, a dispersant, a stabilizer, a coloring agent, and the like according to a general method.

Examples of the excipient include starch and derivatives thereof (dextrin, carboxylmethyl starch, and the like), cellulose and derivatives thereof (methyl cellulose, hydroxypropyl methyl cellulose, and the like), saccharides (lactose, white sugar, glucose, trehalose, and the like), citric acid and salt thereof, malic acid and salts thereof, and ethylenediamine tetraacetic acid and salts thereof.

Examples of the binder include starch and derivatives thereof (alphanized starch, dextrin, and the like), cellulose and derivatives thereof (ethyl cellulose, carboxymethyl cellulose sodium, hydroxypropyl methyl cellulose, and the like), gum Arabic, tragacanth, gelatin, saccharides (glucose, ethanol, white sugar, and the like), ethanol, and the like.

Examples of the disintegrating agent include starch and derivatives thereof (carboxymethyl starch, hydroxypropyl starch, and the like), cellulose and derivatives thereof (carboxymethyl cellulose sodium, crystalline cellulose, hydroxypropyl methyl cellulose, and the like), carbonate salts (calcium carbonate, calcium hydrogen carbonate, and the like), tragacanth, gelatin, agar, and the like.

Examples of the lubricant include stearic acid, calcium stearate, magnesium stearate, talc, titanium oxide, calcium hydrogen phosphate, dried aluminum hydroxide gel, a sucrose fatty acid ester, edible fat and oil, and the like.

Examples of the preservative include paraoxybenzoate esters, sulfite salts (sodium sulfite, sodium pyrosulfite, and the like), phosphate salts (sodium phosphate, calcium polyphosphate, sodium polyphosphate, sodium metaphosphate, and the loke), dehydroacetic acid, sodium dehydroacetate, glycerin sorbate, saccharides, and the like.

Examples of the antioxidant include sulfite salts (sodium sulfite, sodium hydrogen sulfite, and the like), erythorbic acid, L-ascorbic acid, cysteine, thioglycerol, butylhydroxyanisole, dibutylhydroxytoluene, propyl gallate, ascorbyl palmitate, dl-α-tocopherol, and the like.

Examples of the tonicity agent include sodium chloride, sodium nitrate, potassium nitrate, dextrin, glycerin, glucose, and the like.

Examples of the buffer solution include sodium carbonate, hydrochloric acid, boric acid, phosphate salts (sodium hydrogen phosphate and the like), and the like.

Examples of the coating agent include cellulose derivatives (hydroxypropyl cellulose, cellulose acetate phthalate, hydroxypropylmethyl cellulose phthalate, and the like), shellac, polyvinylpyrrolidone, polyvinyl pyridines (poly-2-vinylpyridine, poly-2-vinyl-5-ethylpyridine, and the like), polyvinyl acetyldiethyl aminoacetate, polyvinyl alcohol phthalate, methacrylate-methacrylic acid copolymer, and the like.

Examples of the flavoring agent include saccharides (glucose, white sugar, lactose, and the like), saccharin sodium, sugar alcohols, and the like.

Examples of the solubilizer include ethylene diamine, nicotinic acid amide, saccharin sodium, citric acid, citrate salts, sodium benzoate, polyvinylpyrrolidone, polysorbates, sorbitan fatty acid esters, glycerin, polyprene glycol, benzyl alcohols, and the like.

Examples of the base include fats (pork fat and the like), vegetable oil (olive oil, sesame oil, and the like), animal oil, lanolin acid, vaseline, paraffin, resin, bentonite, glycerin, glycol oil, and the like.

Examples of the dispersant include gum Arabic, tragacanth, cellulose derivatives (methyl cellulose, and the like), sodium alginate, polysorbates, sorbitan fatty acid esters, and the like.

Examples of the stabilizer include sulfite salts (sodium hydrogen sulfite and the like), nitrogen, carbon dioxide, and the like.

The total content (content obtained by adding up the contents of all effective components contained in the composition) of the effective components in the food composition and/or the pharmaceutical composition differs depending on the conditions such as the types, components, and forms of food and medicine etc., and can be properly selected without being particularly limited within a range in which the effect of the present invention can be obtained.

In particular, when the form of the food composition and/or the pharmaceutical composition according to the embodiment is a tablet, the total content of the effective components in the tablet is not particularly limited within a range in which the effect of the present invention can be obtained, but the total content in terms of dry weight of the effective components in the total mass of the food composition and/or pharmaceutical composition is preferably 20% by mass or more and more preferably 50% by mass or more. Also, the total content may be 100% by mass or less and is preferably 99% by mass or less.

In the present invention, the total intake amount of the effective components is not particularly limited and is properly selected according to the types and components of food and medicine, etc. For example, the total intake amount in terms of dry weight of effective components per day for every adult is preferably 0.01 g or more, more preferably 0.03 g or more, and preferably 10 g or less, more preferably 4 g or less.

### EXAMPLES

The present invention is described in further detail by giving examples below. The scope of the present invention is not limited to these examples.

### Test Example 1

The functions of spirulina, phycocyanin, the spirulina enzymatically degraded product, and the phycocyanin enzymatically degraded product on muscle and muscle atrophy were evaluated by using a sciatic neurectomized model rat as a sarcopenia model.

### <Test Substance>

Spirulina, phycocyanin, the spirulina enzymatically degraded product, and the phycocyanin enzymatically degraded product were used as test substances.

### <<Spirulina>>

Spirulina platensis was proliferated under basic conditions (pH 11) in an outdoor culture pond. Next, 50 g of the alga powder obtained by spray-drying the proliferated spirulina platensis was suspended in 500 mL of distilled water in an autoclave and subjected to extraction by adjusting the pressure at an extraction temperature of 120° C. for 1 hour.

The resultant extracted solution was adjusted to pH 4.0 with citric acid. Then, alga residue and proteins (insoluble fraction) were removed by centrifugal separation, producing a spirulina extract which was a spirulina hot-water extracted solution.

The resultant spirulina extract was spray-dried and then crushed to produce powdery spirulina.

### <<Phycocyanin>>

First, 65 kg of spirulina dried alga (spray dried product) produced in an outdoor culture tank was added to 1300 L of a 1% calcium chloride (anhydrous) solution, and stirred for 15 minutes, preparing a more uniform suspension. Then, phycocyanin in cyanobacteria was extracted in the solution under standing conditions at 20° C. for 15 hours, producing an extracted solution.

Then, 32 kg of sodium dihydrogen phosphate was added to the extracted solution, stirred for 0.5 hours, and then reacted under standing at 20° C. for 2.5 hours. Consequently, calcium phosphate was produced, and impurities of phycocyanin were adsorbed on the calcium phosphate, producing an adsorbed substance. Then, the extracted solution was led to a centrifugal separator and centrifuged for 15 minutes at a gravitational acceleration of 10,000 G, thereby removing the residue of cyanobacteria and the adsorbed substance from the extracted solution. Then, a low-molecular component and salts were removed from the phycocyanin extracted solution by ultrafiltration using a separation membrane with a cut-off molecular weight of 10,000. Then, trehalose and trisodium citrate were added and mixed, and the resultant mixture was spray-dried to obtain 15 kg of phycocyanin pigment dried product. This was used as phycocyanin. The content of phycocyanin in 100% by mass of the phycocyanin pigment powder was about 30% by mass (C-phycocyanin: about 22% by mass, allophycocyanin: about 8% by mass).

### <<Spirulina Enzymatically Degraded Product>>

First, 100 g of the spirulina was dissolved in 1400 mL of distilled water, and then heated to 50° C. to 52° C. Next, the aqueous solution was adjusted to pH 7.0 by adding a 1N sodium hydroxide solution. Then, 2% by mass of protein (SD-NY10, manufactured by Amano Enzyme Inc.) relative to the protein in spirulina was added to the aqueous solution and then stirred at 50° C. to 52° C. for 6 hours, thereby effecting enzymatic degradation of spirulina. The solution after reaction was cooled to room temperature, led to a centrifugal separator, and then centrifuged for 15 minutes at a gravitational acceleration of 10,000 G. The supernatant was collected and then freeze-dried to obtain 62 g of spirulina enzymatically degraded product which was a spirulina protease decomposition product.

### <<Phycocyanin Enzymatically Degraded Product>>

First, 17 g of the phycocyanin was dissolved in 1400 mL of distilled water, and then heated to 50° C. to 52° C. Next, the aqueous solution was adjusted to pH 7.0 by adding 1N hydrochloric acid. Then, 2% by mass of protein (SD-NY10, manufactured by Amano Enzyme Inc.) relative to phycocyanin was added to the aqueous solution and then stirred at 50° C. to 52° C. for 6 hours, thereby effecting enzymatic degradation of phycocyanin. The solution after reaction was cooled to room temperature, led to a centrifugal separator, and then centrifuged for 15 minutes at a gravitational acceleration of 10,000 G. The supernatant was collected and then freeze-dried to obtain 16 g of phycocyanin enzymatically degraded product which was a phycocyanin protease decomposition product.

### <Formation of Sciatic Neurectomized Model>

Slc: SD male rat (Japan SLC Inc.) was used. The sciatic neurectomized model rat as a sarcopenia model was prepared as described below.

After the 14th day administration of the test substances, the left hind leg of the rat was shaved, and then an incision was made in the left thigh part. The muscle was divided along the muscle fibers to expose the sciatic nerve. The sciatic nerve was removed by 1 cm, and then the incision part was sutured, preparing a sciatic neurectomized model rat.

### <Test System>

The experiment groups, test substances, dosage amounts, treatment, and number of cases are shown in Table 1 below. With respect to the number of times of administration and the administration period, oral administration was performed for 21 days at a frequency of once a day (amount of administration solution: 10 mL/kg). In addition, the A1 group is also referred to as “Sham group” (general rat group not sciatic neurectomized), and A2 group is also referred to as a “medium group”.

TABLE 1

| | | Dosage | | |
|---|---|---|---|---|
| Experiment | | amount | | Number |
| group | Test substance | (mg/kg) | Treatment | of cases |
| A1 (Sham | Water for injection | — | Sham | 4 |
| group) | (medium) | | | |
| A2 (medium | Water for injection | — | Sciatic | 6 |
| group) | (medium) | | neurectomized | |
| A3 | Spirulina | 750 | Sciatic | 6 |
| | | | neurectomized | |
| A4 | Spirulina | 750 | Sciatic | 6 |
| | enzymatically | | neurectomized | |
| | degraded product | | | |
| A5 | Phycocyanin | 750 | Sciatic | 6 |
| | enzymatically | | neurectomized | |
| | degraded product | | | |
| A6 | Phycocyanin | 120 | Sciatic | 6 |
| | | | neurectomized | |

### <Measurement of Muscle Mass>

In the A1 to A6 groups, the gastrocnemius muscle and soleus muscle of both hind legs were removed on the 21th day of administration of the test substances, and the wet weights (g) were measured.

In the A1 to A6 groups, the muscle mass (wet weight (g) of each of the muscles of both hind legs/body weight (g), % by weight) was calculated.

Also, the average muscle mass of each of the muscles in each of the A3 to the A6 groups was compared with the A2 group (medium group), and the rate of increase (%) in muscle mass in each of the A3 to A6 groups was calculated (average muscle mass (% by weight) of each muscle of both hind legs in each of the A3 to A6 groups)/(average muscle mass (% by weight) of each muscle of both hind legs in the A2 group (medium group)×100).

Further, the rate of change (%) in muscle mass in each of the A1 to A6 groups was calculated (average wet weight (g) of each muscle of left hind leg)/average wet weight (g) of each muscle of right hind leg×100, % by weight).

### <DNA Microarray>

RNA was extracted from the resection side gastrocnemius muscle of each of the A1 to A3 groups and the A6 group and used for a DNA microarray. DNA microarray was performed as follows.

In each of the groups, an RNA sample was pooled, and CDNA synthesis and Cy3-labelled CRNA synthesis and purification were performed by using Low Input Quick Amp Labelling Kit (Agilent). The concentration of the resultant labelled CRNA and the Cy3 incorporation were calculated from the absorbances at 260 nm, 280 nm, 550 nm, and 320 nm, and confirmed to satisfy the reference value (Cy3-CTP incorporation>6 pmol/μg). Then, each labelled CRNA was fragmented by using Gene Expression Hybridization Kit (Agilent), applied to Whole Mouse Genome Microarray Ver 2.0 (Agilent), and hybridized at 65° C. for 17 hours. Next, the array slide was washed by using Gene Expression Wash Buffer 1 and 2 (Agilent). An array image obtained by scanning with a microarray scanner was digitized by using array analysis software GenePix Pro (Molecular Devices). The fluorescence strength value was normalized, and the ratio of each of the groups to the Sham group was calculated.

### <Measurement Result of Muscle Mass>

The results of the rate of increase in muscle mass are shown in Table 2.

TABLE 2

| | | Rate of increase in muscle | | Rate of increase in | |
|---|---|---|---|---|---|
| | | mass of left hind leg (sciatic | | muscle mass of right | |
| | | neurectomized) [%] | | hind leg [%] | |
| Experiment | Test | Gastrocnemius | Soleus | Gastrocnemius | Soleus |
| group | substance | muscle | muscle | muscle | muscle |
| A3 | Spirulina | 98.5 | 92.6 | 98.7 | 95.0 |
| A4 | Spirulina | 98.2 | 103.7 | 97.4 | 97.5 |
| | enzymatically | | | | |
| | degraded | | | | |
| | product | | | | |
| A5 | Phycocyanin | 99.6 | 100.0 | 102.5 | 100.0 |
| | enzymatically | | | | |
| | degraded | | | | |
| | product | | | | |
| A6 | Phycocyanin | 102.0 | 100.0 | 99.7 | 105.0 |

In the A6 group administered with phycocyanin, the rate of increase in muscle mass of the gastrocnemius muscle in the left hind leg (sciatic neurectomized) was 102.0%, and thus the muscle mass was increased.

In the A4 group administered with the spirulina enzymatically degraded product, the rate of increase in muscle mass of the soleus muscle in the left hind leg (sciatic neurectomized) was 103.78, and thus the muscle mass was increased.

In the A5 group administered with the phycocyanin enzymatically degraded product, the rate of increase in muscle mass of the gastrocnemius muscle in the right hind leg was 102.5%, and thus the muscle mass was increased.

In the A6 group administered with phycocyanin, the rate of increase in muscle mass of the soleus muscle in the right hind leg was 105.0%, and thus the muscle mass was increased.

The results of the rate of change are shown in Table 3.

TABLE 3

| | | Rate of change [% by weight] | |
|---|---|---|---|
| Experiment | | Gastrocnemius | Soleus |
| group | Test substance | muscle | muscle |
| A1 (Sham | Water for injection | 101.5 | 101.3 |
| group) | (medium) | | |
| A2 (medium | Water for injection | 75.2 | 67.4 |
| group) | (medium) | | |
| A3 | Spirulina | 75.0 | 66.1 |
| A4 | Spirulina enzymatically | 75.8 | 71.7 |
| | degraded product | | |
| A5 | Phycocyanin enzymatically | 73.1 | 69.4 |
| | degraded product | | |
| A6 | Phycocyanin | 77.1 | 63.8 |

The rate of change in each of the gastrocnemius muscle and the soleus muscle of a sarcopenia model rat in the A2 group (medium group) was significantly decreased as compared with a usual rat in the A1 group (Sham group).

When the values of rate of change in each of the A3 to A6 groups administrated with the test substance are increased as compared with the value of rate of change in the A2 group, the muscle mass is considered to be increased by the test substances.

In the gastrocnemius muscle, the rate of change in the A4 group administrated with the spirulina enzymatically degraded product and the rate of change in the A6 administrated with phycocyanin were increased as compared with the A2 group.

In the soleus muscle, the rate of change in the A4 group administrated with the spirulina enzymatically degraded product and the rate of change in the A5 group administrated with the phycocyanin enzymatically degraded product were increased as compared with the A2 group.

### <Results of DNA Microarray>

<<Gene Estimated to be Associated with the Increase in Muscle Mass and the Suppression of Decrease in Muscle Mass>>

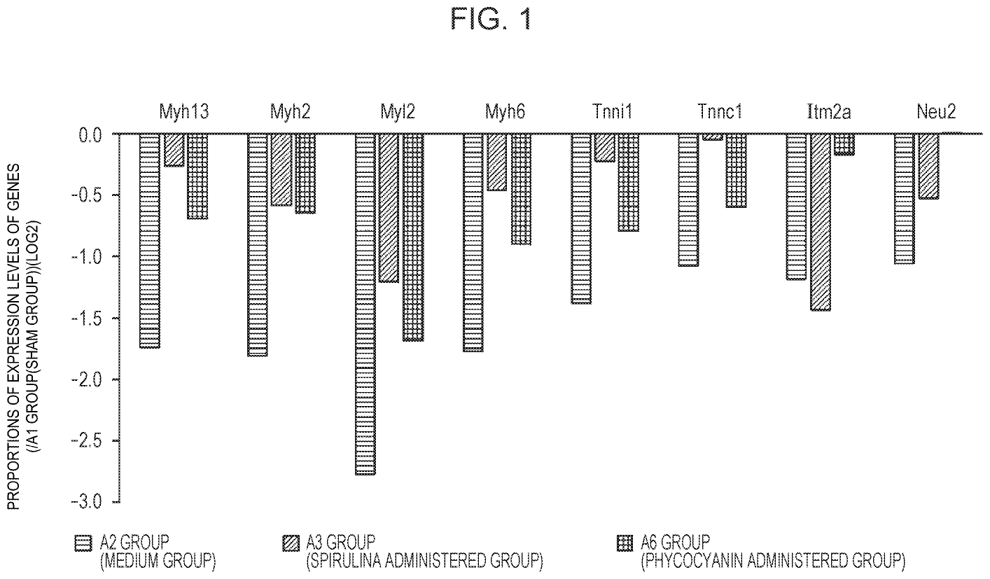

FIG. 1 shows the results of the expression levels of genes estimated to be associated with the increase in muscle mass and the suppression of decrease in muscle mass.

FIG. 1 is a graph showing the ratios of the expression levels of genes estimated to be associated with the increase in muscle mass and the suppression of decrease in muscle mass of the gastrocnemius muscle relative to the A1 group (Sham group).

As shown in FIG. 1, it was recognized that in the gastrocnemius muscle, the administration of spirulina or phycocyanin alleviates, in comparison with the A2 group (medium group), the suppression of expression of Myh13, Myh2, My12, and Myh6 associated with the production of myosin, which is a muscle structural protein, and Tnni1 and Tnnc1 associated with the production of troponin, which is a muscle structural protein. Also, alleviation was recognized in the suppression of expression of Itm2a as a muscle satellite cell marker associated with myogenesis and Neu2 associated with myoblast differentiation. Therefore, it was recognized that spirulina and phycocyanin have the function of increasing muscle mass and the function of suppressing a decrease in muscle mass. Also, it was supposed from these results that the spirulina enzymatically degraded product and the phycocyanin enzymatically degraded product similarly exhibit the function of increasing muscle mass and the function of suppressing a decrease in muscle mass.

<<Gene Estimated to be Associated with Muscle Atrophy>>

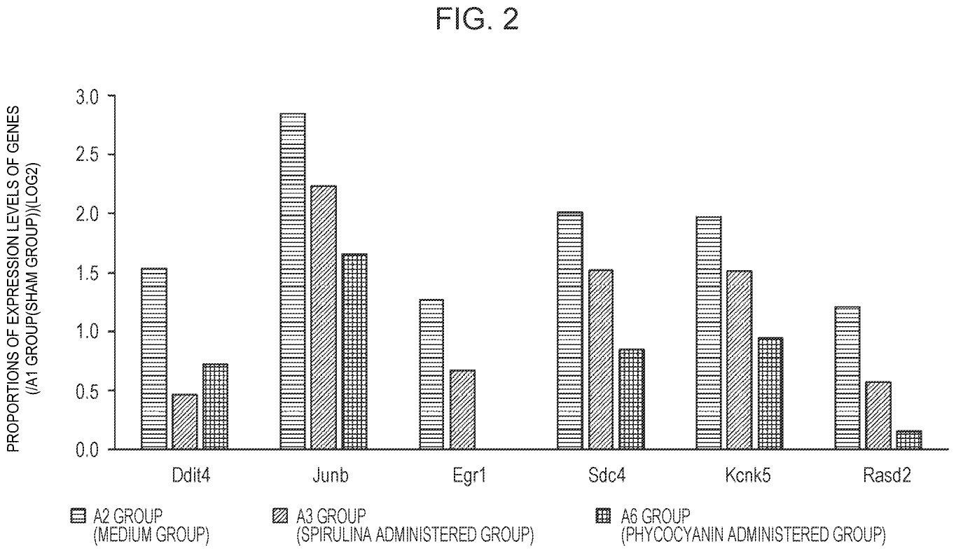

FIG. 2 shows the results of the expression levels of genes associated with muscle atrophy.

FIG. 2 is a graph showing the ratios of the expression levels of genes estimated to be associated with muscle atrophy in the gastrocnemius muscle relative to the A1 group (Sham group).

As shown in FIG. 2, it was recognized that in the gastrocnemius muscle, the administration of spirulina or phycocyanin alleviates, in comparison with the A2 group (medium group), the enhancement of expression of Ddit4, Junb, and Egr1 associated with muscle atrophy and muscle regeneration and Sdc4, Kcnk5, Rasd2 associated with muscle regeneration. Therefore, it was recognized that spirulina and phycocyanin have the function of suppressing muscle atrophy. Also, it was supposed from these results that the spirulina enzymatically degraded product and the phycocyanin enzymatically degraded product similarly exhibit the function of suppressing muscle atrophy.

The results of measurement of muscle mass and the results of the DNA microarray recognized that a composition containing as an effective component at least one selected from the group consisting of spirulina, phycocyanin, the spirulina enzymatically degraded product, and the phycocyanin enzymatically degraded product is a composition exhibiting the function of increasing muscle mass, the function of suppressing a decrease in muscle mass, and the function of suppressing muscle atrophy.

1. A composition for increasing muscle mass and suppressing a decrease in muscle mass, comprising as an effective component at least one selected from the group consisting of spirulina, phycocyanin, a spirulina enzymatically degraded product, and a phycocyanin enzymatically degraded product.

2. The composition according to claim 1, wherein the spirulina enzymatically degraded product and the phycocyanin enzymatically degraded product are obtained by enzyme action of protease.

3. A composition for increasing muscle mass and suppressing muscle atrophy, comprising as an effective component at least one selected from the group consisting of spirulina, phycocyanin, a spirulina enzymatically degraded product, and a phycocyanin enzymatically degraded product of phycocyanin.

4. The composition according to claim 3, wherein the enzyme for the spirulina enzymatically degraded product and the phycocyanin enzymatically degraded product are obtained by enzyme action of protease.

5. The composition according to claim 1, wherein the composition is a food composition.

6. The composition according to claim 5, wherein the composition is a health food, a functional food, a nutritional supplement food, a supplement, a food with a health claim, a food for specialized health uses, a functional nutritional food, a food with function claim, or a food for patients.

7. The composition according to claim 1, wherein the composition is a pharmaceutical composition.

8. The composition according to claim 7, wherein the composition is a pharmaceutical composition used for curing, preventing, or improving sarcopenia or locomotive syndrome.

9. The composition according to claim 3, wherein the composition is a food composition.

10. The composition according to claim 9, wherein the composition is a health food, a functional food, a nutritional supplement food, a supplement, a food with a health claim, a food for specialized health uses, a functional nutritional food, a food with function claim, or a food for patients.

11. The composition according to claim 3, wherein the composition is a pharmaceutical composition.

12. The composition according to claim 11, wherein the composition is a pharmaceutical composition used for curing, preventing, or improving sarcopenia or locomotive syndrome.
